(19) United States
(12) Patent Application Publication
(10) Pub. No.: US 2015/0338401 A1
(43) Pub. Date: Nov. 26, 2015

(54) MULTIPLEXED BIOASSAY TECHNIQUES

(71) Applicants: Ayal RAM, Ramat Gan (IL); LIGHTSTAT, LLC, Southlake, TX (US)

(72) Inventor: Ayal Ram, Ramat Gan (IL)

(21) Appl. No.: 14/443,504

(22) PCT Filed: Nov. 18, 2013
(86) PCT No.: PCT/US2013/070528

§ 371 Date: May 18, 2015

Related U.S. Application Data
(60) Provisional application No. 61/796,650, filed on Nov. 16, 2012.

(51) Int. Cl. G01N 33/543

(52) U.S. Cl. CPC G01N 33/54373; G01N 33/54393

(57) ABSTRACT

Techniques for multiplexed bioassays include a substrate in which is formed a microchannel in fluid communication between an entry port and an exit port. A first portion of the microchannel is configured to supply multiple different labeled probes, each selected to complex with one of a corresponding plurality of different analytes. A second portion of the microchannel comprises multiple corresponding different supplementary probes covalently bound to the substrate. A supplementary probe is selected to bind to a part of a corresponding analyte, which part is exposed when the corresponding analyte is complexed with a corresponding labeled probe. The techniques include a sensor configured to detect signals emitted from the labeled probes in the second portion of the microchannel.

### CROSS-REFERENCE TO RELATED APPLICATIONS

This application is a 371 national stage of PCT/US2013/070528 filed Nov. 18, 2013, which claims the benefit of U.S. Provisional Application No. 61/796,650 filed Nov. 16, 2012, the entire contents of which are hereby incorporated by reference in their entirety.

### BACKGROUND OF THE INVENTION

Multiplexed, sensitive, and on-chip molecular diagnostic assays are useful in both clinical and research settings. One method uses the binding of specific protein molecules to probes which have a very high specificity to that protein by use of immunochemical techniques. These techniques are continuously being developed to determine the presence of specific proteins in biological fluids. The analytical technique often involves the immobilization of the protein of choice to its specific probe, which is then measured using a variety of signaling technologies. The more sensitive the signaling process, the more accurate and precise is the immunochemical method. Still, many proteins are in such small quantities that the immunochemical procedure is not sensitive enough to detect them. Many detection strategies employ amplification schemes to achieve sensitivity by labeling surface bound targets for which measurements can be accumulated over long integration times. In standard amplification reactions such as the commercially available enzyme-linked immunosorbent assay (ELISA), enzyme-assisted amplification reactions occur on microplates with net volumes on the order of 100 □1 and are still considered the gold standard for protein detection.

While suitable for many purposes, the immunochemical techniques available today are time consuming and require a multi-step analytical procedure. Also these procedures do not easily lend themselves to an analytical procedure by the bedside, since they require complex, large instrumentation and infrastructure. Furthermore, the present procedures cannot simultaneously determine many proteins as each requires a specific test procedure.

### SUMMARY OF THE INVENTION

It is herein recognized that it is advantageous to perform the simultaneous analysis of minute quantities of multiple protein entities using a miniature disposable device, which can be carried out in the absence of fixed or large instrumentation or infrastructure, or some combination.

In a first set of embodiments, an apparatus includes a substrate in which is formed a microchannel in fluid communication between an entry port and an exit port. A first portion of the microchannel is configured to supply multiple different labeled probes, each selected to complex with one of a corresponding plurality of different analytes. A second portion of the microchannel includes multiple corresponding different supplementary probes covalently bound to the substrate. A supplementary probe is selected to bind to a part of a corresponding analyte, which part is exposed when the corresponding analyte is complexed with a corresponding labeled probe. The apparatus further includes a sensor configured to detect, in the second portion of the microchannel, signals emitted from the labeled probes.

In a second set of embodiments, a method includes providing a microfluidic device comprising a sensor and substrate that includes a microchannel in fluid communication between an entry port and an exit port. A first portion of the microchannel is configured to supply multiple different labeled probes, each selected to complex with one of a corresponding multiple different analytes. A second portion of the microchannel includes multiple corresponding different supplementary probes covalently bound to the substrate. A supplementary probe is selected to bind to a part of a corresponding analyte, which part is exposed when the corresponding analyte is complexed with a corresponding labeled probe. The sensor is configured to detect signals emitted from the labeled probes in the second portion of the microchannel. The method also includes moving a sample fluid from the entry port to the exit port. The method further includes obtaining data from the sensor that indicates measurements of the signals emitted from labeled probes bound to the supplementary probes in the second portion during an observation period.

In a third set of embodiments, a kit includes a device comprising a sensor and a substrate in which is formed a microchannel in fluid communication between an entry port and an exit port. A first portion of the microchannel is configured to supply a plurality of different labeled probes, each selected to complex with one of a corresponding plurality of different analytes. A second portion of the microchannel configured for covalently binding a plurality of corresponding different supplementary probes to the substrate. A supplementary probe is selected to bind to a part of a corresponding analyte, which part is exposed when the corresponding analyte is complexed with a corresponding labeled probe. The kit also includes a supply of a plurality of molecules for each of the plurality of different labeled probes.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a block diagram that illustrates an example apparatus for a multiplexed bioassay device, according to an embodiment;

FIG. 2A and FIG. 2B are block diagrams that illustrate example label portions of a microchannel for a multiplexed bioassay device, according to various embodiments;

FIG. 3 is a block diagrams that illustrates an example fix portion of a microchannel for a multiplexed bioassay device, according to an embodiment;

FIG. 4 is a flow diagram that illustrates an example method for performing a multiplexed bioassay using the device of FIG. 1, according to an embodiment;

FIG. 5 is a block diagram that illustrates a computer system upon which an embodiment of the invention may be implemented; and

FIG. 6 illustrates a chip set upon which an embodiment of the invention may be implemented.

### DETAILED DESCRIPTION

A method and apparatus are described for multiplexed bioassays. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of three protein analytes and fluorescence measurements; but, embodiments are not limited to this context. In other embodiments, more or fewer analytes, or other analytes are assayed using other measurement techniques, including luminesce, polarization, quantum dots, Förster resonance energy transfer (FRET), amplified colorimetric outputs using enzymes and substrates, among others, alone or in some combination. In some embodiments, the analytes are nucleic acids, such as DNA and RNA molecules and nucleic acid sequences, and the labeled probes and supplementary probes are selected to bind to different parts of the analyte, such as nucleic acid probes with complementary sequences to two or more parts of the analyte nucleic acid.

As used herein, a microfluidic channel has at least one dimension in a size range from about 0.1 micron to about 1000 microns (1 micron, also called a micrometer, □m, =10⁻⁶ meters). Similarly, a microstructure has a greatest dimension in a range from about 1 micron to about 1000 microns.

The bioassay is performed. on a sample from a subject. In various embodiments, the subject is an animal, typically a mammal, preferably a human, and the biological sample is selected from the group consisting of hair, nails, serum, blood, peripheral blood, plasma, sputum, saliva, mucosal scraping, urine, pleural effusion fluid, cerebrospinal fluid, bone marrow, tissue biopsy, isolated cells or cell suspension on a scaffolding dissolved or suspended in aqueous or non-aqueous solution. In some illustrated embodiments, the analytes are proteins (such as antigens that stimulate a subject's immune system) and the probes are antibodies.

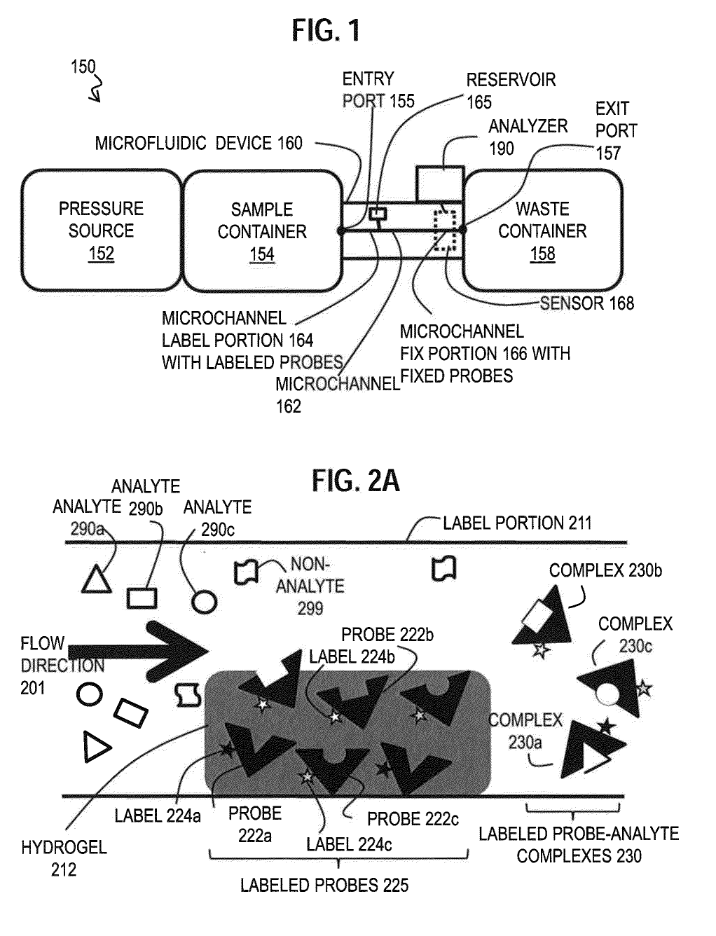

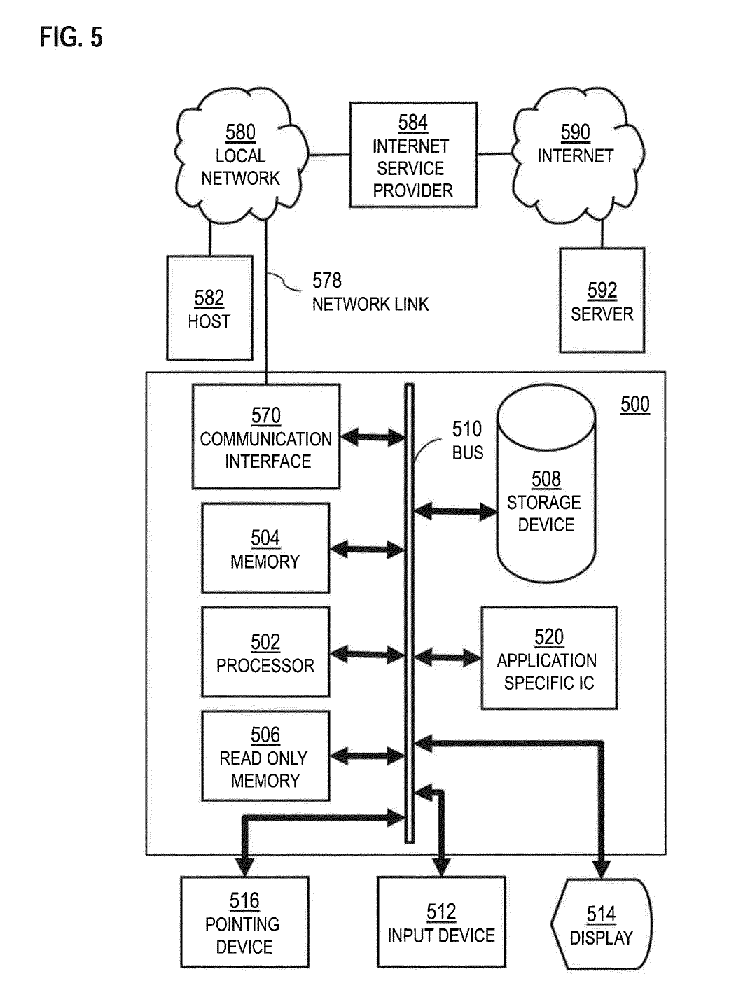

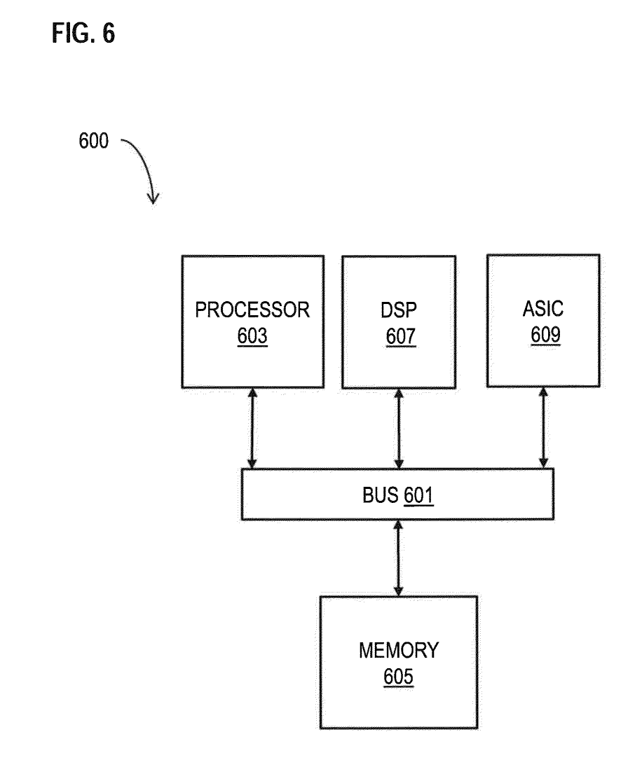

FIG. 1 is a block diagram that illustrates an example apparatus for a multiplexed bioassay device, according to an embodiment. The apparatus 150 includes a microfluidic device 160 having one or more microchannels 162 in which there is a label portion 164 and a fix portion 166. The product of reactions in the microstructures, such as one or more colorimetric or fluorescent products, is observed by sensor 168, such as a photodetector or photodetector array, e.g. a charge coupled device (CCD) array. In some embodiments, the sensor 168 includes an optical source for exciting fluoresce, or optical components such as lenses and mirrors, or some combination. If the device is not transparent to the observations, then the device 160 includes an observation port (not shown) between the sensor 168 and the fix portion 166 of the microchannel 162. Analog or digital data from the sensor is analyzed by analyzer 190 to determine existence or relative or absolute quantity of two or more analytes or some combination. In various embodiments, the analyzer 190 includes a computer system as described below with reference to FIG. 5 or a chip set as described below with reference to FIG. 6. In some embodiments, the analyzer 190 is a chip set 600 that is part of the microfluidic device 160.

Sample fluid is introduced into the microchannel 162 from a sample container 154 (e.g., pipette or absorbent pad). The fluid is propelled through the microchannel by a motive force, such as gravity, a micropump in the microchannel 162 or elsewhere in the microfluidic device 160, or by an external pressure source 152. Fluid passes out of each container 154 into the microchannel 162 through an entry port 155 (e.g., pipette needle) in the device 160. The fluid that passes through the microchannel exits through an exit port 157 and is ejected into a waste container 158. In some embodiments, the waste container is also part of the device 160. Thus described is a microfluidic device comprising a substrate in which is formed a microchannel in fluid communication between an entry port and an exit port. The pressure source or micro-pump constitutes an actuator configured to move a sample fluid from the entry port to the first portion of the microchannel and then to the second portion of the microchannel and then to the exit port.

In some embodiments, the labeled probes in the first portion are provided by a reservoir in the first portion, such as reservoir 165. In the illustrated embodiments, the labeled probes are inserted into the microchannel embedded in a hydrogel. In these embodiments, the hydrogel dissolves or dissociates in the presence of the sample fluid or in response to strong enough flow of the sample fluid. The dissolution or dissociation of the hydrogel releases the labeled probes. In some embodiments, the labeled probes are mixed with the hydrogel and then deposited into the microchannels by placing a drop of the hydrogel-labeled probe mixture in the channel and then freeze drying the hydrogel so that it adheres to the microchannel.

In some embodiments, the fixed probes in the second portion are provided by a reservoir in the second portion (not shown), and include a component to covalently bind to material in or on a substrate of the device in the second portion. In some embodiments, the fixed probes are covalently bound to the microchannel substrate during fabrication. Typically, the approach includes surface silanization followed by anchoring to a functional group of a silanizing agent. PDMS (polydimethylsiloxane), a silicon-based organic polymer, attained widespread use because of the low cost, rapid and prototype-friendly fabrication, as well as optical transparency, malleability, and gas permeability (appropriate for some applications). Recently, plastic substrates such as Poly(methyl methacrylate (PMMA), polystyrene (PS), and cyclic olefin copolymer (COC) have gained attention owing to low cost of fabrication (e.g., injection molding or hot embossing), a chemical resistance superior to PDMS, optical transparency, and low autofluorescence. PDMS and plastic surfaces are relatively inert and lack functional groups (i.e., sites for protein attachment). Thus, involved chemical surface preparation is generally required to induce surface functional groups for protein immobilization. Any chemical preparation known in the art may be used. For example, naturally or artificially grown oxide on the silicon surface makes silanol-based chemistries compatible with for protein immobilization. PDMS is hydrophobic in native form, so proteins tend to readily and nonspecifically bind to the surface. Therefore, blocking of the adsorptive surface must be done before an assay is completed. PDMS lacks functional groups for covalent derivatization. Silanol groups can be introduced after oxygen plasma treatment. Other approaches include a strategy to covalently photopattern 3-D hydrogel plugs with functionalized protein G inside microfluidic channels on a hydrophilic PDMS substrate coated with polyelectrolyte multilayers (PEMS). Another approach includes an oxidation reaction in acidic H2O2 solution and a sequential silanization reaction using neat silane reagents for surface modification of PDMS.

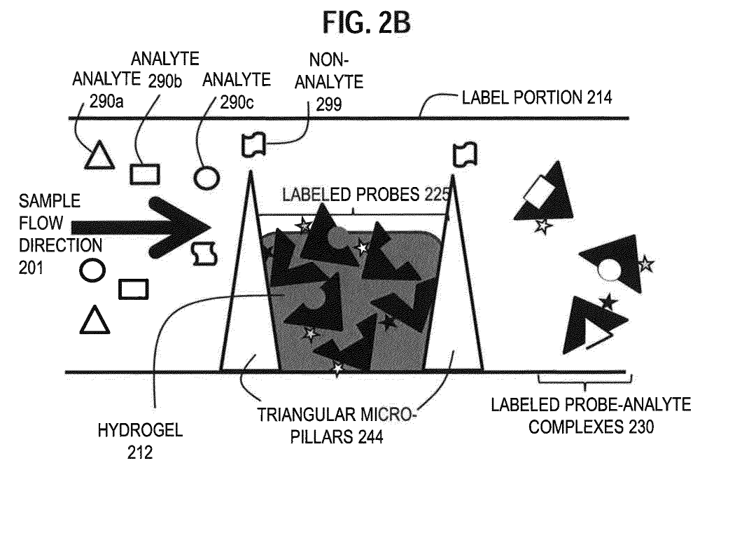

FIG. 2A and FIG. 2B are block diagrams that illustrate example label portions of a microchannel for a multiplexed bioassay device, according to various embodiments. FIG. 2A is a block diagram that illustrates an example label portion 211 of a microchannel without micro-pillars. This is an elevation view cross section with the top and bottom of the diagram indicating the top and bottom of the label portion 211 of a microchannel, such as portion 164 of microchannel 162. Items depicted in this cross section extend or are repeated some distance perpendicular to the page. Label portion 211 is thus a particular embodiment of label portion 164.

Label portion 211 includes a hydrogel microstructure in which is embedded multiple molecules for each of several different probes, e.g., probes 222a, 222b and 22c, that bind specifically to corresponding different analytes to be detected and quantified, such as different analytes 290a, 290b, 290c, respectively, indicated by a triangle, rectangle and oval, respectively. All molecules of each of the different probes 222a, 222b and 222c are depicted with receptacles for the corresponding shapes—triangle, rectangle and oval, respectively. Although analytes 290a, 290b 290c are depicted for purposes of illustration, those analytes are not part of the device 160 or label portion 211, but instead are in the sample on which the label portion 211 operates.

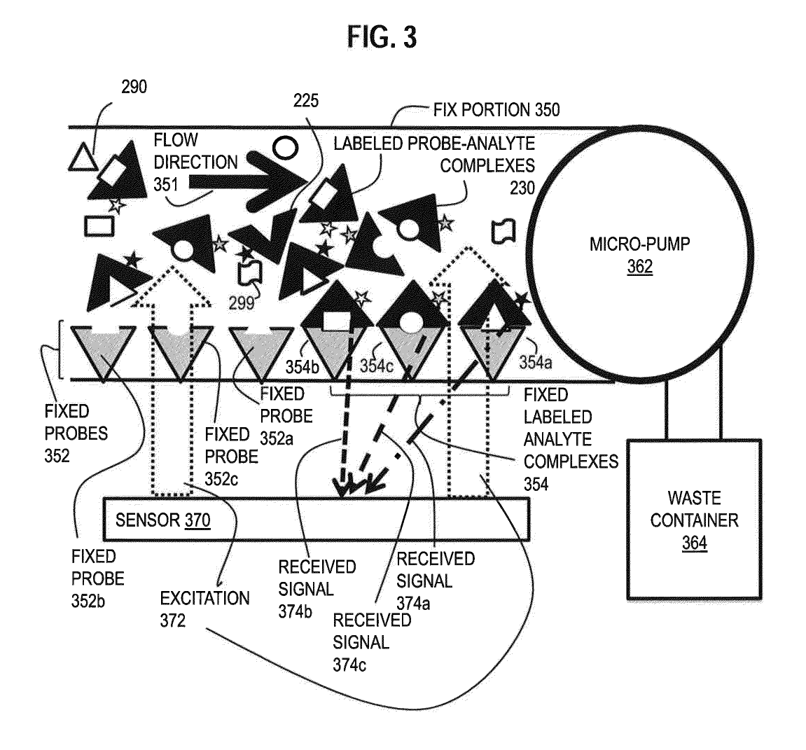

Each different probe includes a different label 224a, 224b, 224c, respectively (represented by stars with different fill—black, white and gray, respectively) that emit corresponding different signals (e.g., different signals 374a, 374b, 374c, respectively (described below with reference to FIG. 3), that can be recorded by sensor 168. The combination of label (224a, 224b, 224c, collectively referenced hereinafter as labels 224) with the corresponding probes (222a, 222b,222c, respectively, collectively referenced hereinafter as probes 222) are called labeled probes and collectively referenced hereinafter as labeled probes 225. Two molecules of each labeled probe 225 are depicted in FIG. 2A, but the hydrogel extends across the width of the microchannel and in practice includes tens to thousands of molecules for each labeled probe. Only three different labeled probes are depicted in FIG. 2A but in other embodiments, more or fewer different labeled probes are included in label portion 211. For example, with fluorescent labels available at over a dozen distinguishable wavelengths, up to a dozen or more different probes can be labeled in some embodiments. In some embodiments, one or more probes are each labeled with different labels as long as the same label is not used for different probes. Thus is described a first portion of the microchannel configured to supply a plurality of different labeled probes, each selected to complex with one of a corresponding plurality of different analytes.

During operation a sample fluid containing one or more analytes and non-analytes (such as non-analyte 299 representing a constituent of the sample, such as a protein, that is not to be detected or quantified by the device 160) is moved through the label portion 211 in flow direction 201. Several molecules of each analyte and non-analyte are depicted in the sample. The flow dissociates or dissolves the hydrogel 212 microstructure, exposing the labeled probes 225 therein. The exposed labeled probes 225 are able to bind to the matching analytes (e.g., 290a, 290b, 290c) to form labeled probe-analyte complexes (e.g., complexes 230a, 230b, 230c, respectively, collectively referenced hereinafter as labeled probe-analyte complexes 230). Although complexes 230 are depicted in FIG. 2A to illustrate operation of the device 160, such complexes are not part of the device 160. Although only one molecule of each different labeled probe-analyte complex is depicted for purposes of illustration, in other embodiments during operation, more or fewer molecules of each different complex are produced. For example, in samples that do not include analyte 290a, complex 230a is not produced.

FIG. 2B is a block diagram that illustrates an example label portion 214 of a microchannel with micro-pillars 244. This is an elevation view cross section with the top and bottom of the diagram indicating the top and bottom of the label portion 214 of a microchannel, such as portion 164 of microchannel 162. Items depicted in this cross section extend or are repeated some distance perpendicular to the page. Label portion 214 is thus a particular embodiment of label. portion 164. The sample flow direction 201, analytes 290a, 290b, 290c, non-analyte 299, labeled probes 225 and labeled probe-analyte complexes 230 are as described above. In this embodiment an array of micro-pillars, such as cylindrical micro-pillars or the triangular micro-pillars 244, are arranged across the microchannel, perpendicular to the page, in one or more rows. The micro-pillars tend to filter out sample constituents, such as red blood cells, that are too big to pass through the spacings between the micro-pillars. Downstream of the appropriate row or rows of micro-pillars, the hydrogel 212 is deposited with the embedded labeled probes 225. The sample flow then dissolves or dissociates the hydrogel, exposing the labeled probes 225 which are then able to bind to the corresponding analytes.

FIG. 3 is a block diagrams that illustrates an example fix portion 350 of a microchannel for a multiplexed bioassay device, according to an embodiment. This is an elevation view cross section with the top and bottom of the diagram indicating the top and bottom. of the fax portion 350 of a microchannel, such as portion 166 of microchannel 162. Items depicted in this cross section extend or are repeated some distance perpendicular to the page. Fix portion 350 is thus a particular embodiment of fax portion 166.

This portion 350 includes fixed probes 352 covalently bound to the substrate of the microchannel, such as fixed probe 352a, fixed probe 352b and fixed probe 352c, selected to bind to exposed parts of the analytes 290a, 290b, 290c, respectively, bound in labeled probe-analyte complexes 230a, 230b, 218c, respectively. Located downstream of the label portion, the fix portion. 350 receives non-analytes 299, unbound analytes 290, unbound labeled probes 225 and labeled probe-analyte complexes 230. Of these constituents, only unbound analytes 290 and labeled probe-analyte complexes 230 can bind to the fixed probes 352. The fixed probes are selected to bind to parts of the analytes that are exposed when the analytes are bound to the labeled probes 225. Thus the fixed probes are also called supplementary probes, to supplement the binding with the labeled probes. Once bound to the fixed (supplementary) probes, the labeled probe-analyte complexes 230a, 230b, 230c form fixed labeled analyte complexes 354a, 354b,354c, respectively, collectively referenced hereinafter as fixed labeled analyte complexes 354. Thus is described a second portion 350 of the microchannel that includes a plurality of corresponding different supplementary probes 352 covalently bound to the substrate. A supplementary probe 352 is selected to bind to a part of a corresponding analyte 290, which part is exposed when the corresponding analyte 290 is complexed with a corresponding labeled probe 225.

Although labeled probes 225, labeled probe-analyte complexes 230, fixed labeled analyte complexes 354, analytes 290 and non-analytes 299 are depicted in FIG. 2A to illustrate operation of the device 160, such complexes are not part of the fix portion 350. Of the depicted molecules, only the fixed probes 352 are originally part of the fix portion 350. Although only one molecule of each different fixed labeled analyte complex 354 is depicted for purposes of illustration, in other embodiments during operation, more or fewer molecules of each different fixed complex is formed. For example, in samples that do not include analyte 290a, fixed complex 524a is not produced.

In the illustrated embodiment, the fixed portion 350 includes a micropump 362. It is advantageous to dispose the micro-pump 362 downstream of the fixed probes 352, so that the micro-pump does not damage the analytes or probes. In some embodiments, the fluid is propelled. otherwise and the micro-pump is omitted. The constituents of the sample that do not bind to the fixed probes are expelled to the waste container 364. In some embodiments, the microchannel 162 is flushed, with a buffer solution to remove unbound constituents from the fixed portion.

Disposed within view of the fixed probes 352 is a sensor 370. In some embodiments, the substrate, in which the microchannel is formed, is transparent to the signals emitted by the labels. In other embodiments, a transparent port for the emitted signals is inserted in the substrate to provide a view of the fixed probes 352. When the unbound labeled probes and labeled probe-analyte complexes are flushed away, the only substantive emissions are from the fixed labeled analyte complexes 354. These emission are received at the sensor 370 as received signals 374a, 374b, 374c, collectively referenced hereinafter as received signal 374, from fixed labeled analyte complexes 354a, 354b, 354c, respectively. The amount of emissions of each type received at sensor 370 is then related to the number of labeled probe-analyte complexes bound to the fixed probes and the efficiency of the corresponding labels 224 associated with each probe 222 in the labeled probes 225 and the affinities of the probes 222 and 352 for the analytes.

In some embodiments, one or more of the emissions received as signals 374 are colorimetric emissions is response to a broadband light source, such as white or ambient light. In some embodiments, one or more of the emissions received as signals 374 are fluorescent emissions in response to one or more excitation signals 372 produced by the sensor 370. In various embodiments, any electromagnetic or radioactive signals are emitted by labels 224 and received by sensor 370. In example embodiments, the signals emitted by labels 224 are in or near the visible portion of the electromagnetic spectrum and referred to as optical signals. Thus is described a sensor configured to detect signals emitted from the plurality of labeled probes in the second portion of the microchannel. In various embodiments, the sensor includes one or more radioactive or electromagnetic or optical detectors, such as a charge coupled device (CCD) array, with zero or more radioactivity, electromagnetic or optical filters to distinguish the different emissions from different labels 224, to produce single pixel or image data responsive to the received signals 374.

In some embodiments, the analyzer 190 receives data from the sensor 370 and derives the presence, relative or absolute amount of one or more analytes, such as analytes 290, based on the amount of each type of received signal, 374, the efficiency of each label 224 to emit based on the ambient or excitation signals, the efficiency of the sensor 370, and the effects, if any, of any optical components, such as lenses, mirrors, optical path lengths, among others, alone or in some combination. The computations are based on the signal emitted or measured for a known standard or a standard curve. When the result is calculated from the measurement of the signal from a standard, it is simple triangulation computation. When the result is to be calculated from a standard curve, the result is taken from the point on the curve corresponding to the signal emitted from the unknown sample concentration.

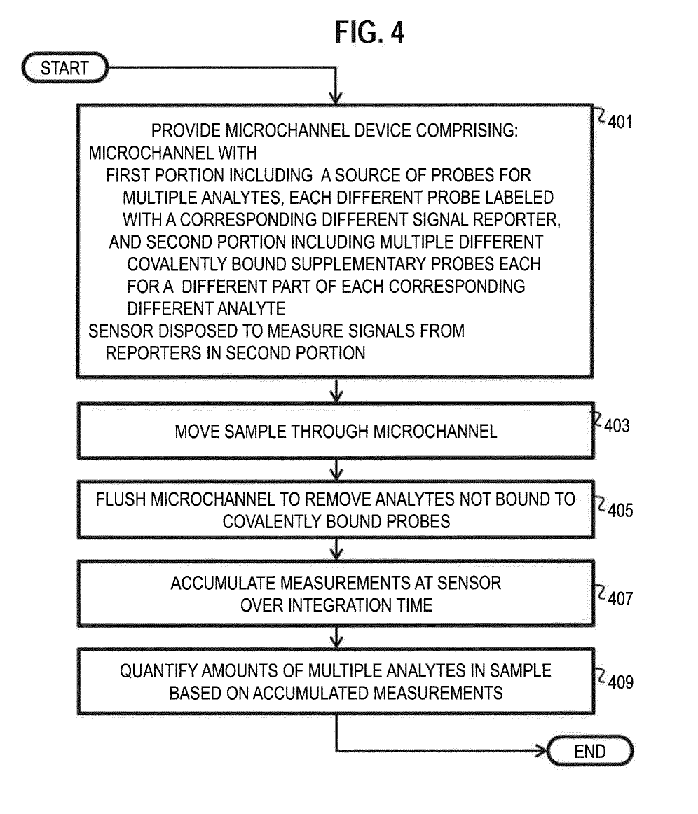

FIG. 4 is a flow diagram that illustrates an example method 400 for performing a multiplexed bioassay using the device of FIG. 1, according to an embodiment. Although steps arc shown as integral blocks in a particular order for purposes of illustration, in other embodiments, one or more steps, or portions thereof, are performed in a different order, or overlapping in time, in series or parallel, or are omitted, or other steps are added, or the process is changed in some combination of ways. For example, in some embodiments, a step is added to flush the microchannels with a buffer solution before step 403 to move a sample fluid through the microchannels.

In step 401, a microfluidic device, such as microfluidic device 160, is provided. In some embodiments, step 401 is achieved by obtaining a preformed device from a supplier. In some embodiments, step 401 includes fabricating the device. In some embodiments, step 401 is performed by one party and the other steps 403 through 409 are performed by one or more different parties.

For example, in some embodiments, straight microchannels are formed in polydimethylsiloxane (“PDMS”) using soft lithography. In some embodiments, channel inlets and outlets are punched, e.g., using a 15-gauge Luer stub; and channels are sonicated in ethanol and dried with argon gas prior to use. Glass slides (VWR, 24×60 mm) to serve as the top of the microchannels are soaked for 1 hour in a 1 M NaOH bath, rinsed with DI water, and dried using argon gas. In sonic embodiments, the PDMS channels and glass slides are plasma-treated (Harrick) on medium RF for 25 seconds, bonded together, and heated at 80 C for 20 minutes In some embodiments, the micro-pump is inserted into a recess, such as a circular recess, formed in the substrate.

A hydrogel (also called aquagel) is a network of polymer chains that are water-insoluble. A polymer is a large molecule (macromolecule) composed of repeating structural units typically connected by covalent chemical bonds. Hydrogels are highly absorbent (they can contain over 99% water) and possess a degree of flexibility due to their significant water content. The pore size is related to the mesh size of the hydrogel microstructure. In the illustrated embodiments, pads made of polyethylene glycol (PEG) or polyethylene glycol diacrylate (PEG-DA) or some combination are photopatterned into one or more microfluidic channels using projection lithography.

In some embodiments, the microstructure hydrogel pads are fabricated affixed to a structural substrate. In various embodiments, the structural substrates are one or more of the following: a glass slide, a PDMS microchannel; a Norland optical adhesive (NOA81) channel; a glass capillary; a thermoplastic polymer chips (such as Zeonex 690R); and, similar structural substrates.

The function of a labeled probe species is to bind to an analyte molecule in a sample and thus bind a label to the analyte. The probe molecule species is often a large biomolecule, such as a protein antibody or a strand of DNA complementary to at least a portion of an analyte DNA strand. In some embodiments, the labelled probes are inserted into the microchannels as a slurry with the hydrogel and then freeze-dried to print them on to the side of the microchannel.

The function of the label is to emit signals that can be detected and distinguished at the sensor 168, such as sensor 370. In some embodiments, the label is a non-fluorescent dye or fluorophore. Non-fluorescent dyes include chlorantine fast green, sirius red and Chicago blue. Colorimetric protein assay methods can be divided into two groups: those involving protein-copper chelation with secondary detection of the reduced copper and those based on protein-dye binding with direct detection of the color change associated with the bound dye. Example fluorophores used in various embodiments include one or more of Hydroxycoumarin, methoxycoumarin, Alexa fluor, aminocournarin, Cy2, FAM, Alexa fluor, Fluorescein FITC, Alexa fluor, Alexa fluor, HEX, Cy3, Alexa fluor, Alexa fluor, R-phycoerythrin (PE), Rhodamine Red-X, Tamara, Cy3.5, Rox, Alexa fluor 568, Red, Texas Red, Alexa fluor 594, Alexa fluor 633, Allophycocyanin, Alexa fluor 633 650, Cy5, Alexa fluor 660, Cy5.5, TruRed, Alexa fluor 680, Cy7.

In embodiments, that use an enzyme-substrate reaction to label the analyte molecule, the labeled probe molecule includes a portion that will bind strongly to the enzyme. For example, exploiting the high affinity of the biotin-streptavidin reaction, the labeled probe molecule is biotinylated in some embodiments. In other embodiments, the labeled probe molecule includes a streptavidin group and the enzyme includes the biotin. Embodiments that use an enzyme-substrate reaction to label the analyte molecule can produce stronger signals than obtained by directly labeling each analyte molecule with a single labeling molecule. Example enzymes include Horseradish peroxidase (HRP).

In embodiments that use enzymes and substrates, example colorimetric substrates used with HRP include: 5-bromo, 4-chloro, 3-indolylphosphate (BCIP)/Nitro-Blue Tetrazolium (NBT); ABTS (2,2′-Azinobis [3-ethylbenzothiazoline-6-sulfonic acid]-diammonium salt); OPD (o-phenylenediarnine dihydrochloride) [HRP]; MB (3,3′,5,5′-tetramethylbenzidine); and, 3-3′ diaminobenzidine tetrachloride. Example colorimetric substrates used with AP include p-Nitrophenyl Phosphate. Example colorimetric substrates used with B-galactosidase include 5-Bromo-4-Chloro-3-Indolyl β-D-Galactopyranoside. Instead of or in addition to colorimetric substrates, fluorescent substrates are used in some embodiments. Example fluorescent substrates used with HRP include amplex red (7-Hydroxy-3H-phenoxazin-3-one 10-oxide) which gets turned over to resorufin—sold by Life Technologies; and QuantaBlu Fluorogenic Peroxidase Substrate—sold by Thermo Scientific. Example fluorescent substrates used with AP include 2′[2-benzothiazoyl]-6′-hydroxybenzothiazole phosphate [BBTP] —sold by Promega. Example fluorescent substrates used with B-galactosidase include Resorufin-□-galactopyranoside (RG□, Life Technologies); fluorescein-di-B-galactopyranoside (FDG, Life Technologies); and, 4-Methylumbelliferyl β-D-Galactopyranoside (MUG) 9H-(1,3-Dichloro-9,9-Dimethylacridin-2-One-7-yl) β-D-Galactopyranoside. In some embodiments, chemiluminescence substrates are used, such as ELISA HRP Substrates Crescendo and Forte from Luminata™; and NovaBright substrates and Galacton Star substrates from Life Technologies™.

The function of a fixed probe species is to bind to supplementary part of an analyte molecule in a sample to fix the analyte molecule to the microchannel. In some embodiments, molecules of a fixed probe species are covalently embedded to a floor of the microchannel as described above or using any method known in the art.

The sensor can be selected from any of the following: electrodes, optical sensors, photo-electric cells, fluorescent sensors, impedance sensors, cell size sensors, turbidity sensors.

In step 403, a sample fluid is moved from the entry port to the exit port. For example a pipette or absorbent pad or drop of blood from a pin prick is contacted to the entry port. The fluid is moved by gravity, capillary action, pressure from a pressure source 152, or by micro-pump 362 downstream of the fixed portion, or some combination. During step 403, the sample flow dissolves or dissociates the hydrogel or otherwise releases the multiple different labeled probes in the label portion 164 to mix with the sample and bind to any of the different corresponding analytes that are present in the sample. As a result, zero or more different labeled probe-analyte complexes (such as complexes 230) are formed. In the fix portion 166, at least some of any analytes (e.g., 290) and complexes (e.g., 230) present bind to the fixed probes (e.g., 352) to form zero or more fixed labeled analyte complexes (e.g., 354).

In step 405, the microchannel is flushed with an aqueous rinse solution that carries away unbound analytes (e.g., 290), non-analytes (e.g., 299), and labeled probe-analyte complexes (e.g., 230). As a result, only the fixed labeled analyte complexes 354, or zero or more non-labeledanalytes, remain in the fix portion 350.

A significant reaction is one in which some signal is obtained which is differentiable from background noise. Typically, this means that the signal divided by the noise generated by the assay is greater than 3—a widely accepted criteria in this field. The target concentration at which this ratio hits 3 is known based on a calibration curve that is generated. In other embodiments, this all differs based on the incubation conditions, the reaction times, and the types of probes/analytes and. the affinities between them. in embodiments that use enzyme substrate reactions as a labeling means, step 405 includes a subsequent filling of the microchannel with substrate to produce fluorescent products that accumulate in the microchannel.

In step 407, a measurement is made of the observable product during the observation period while the fixed labeled analyte complexes are excited.

In step 409, the presence or relative or absolute amount of one or more analytes in the sample is quantified based on the accumulated measurements.

In some embodiments, calibration curves for analytes are determined by using a sample with known amounts of one or more analytes in step 403.

In some embodiments, a kit includes a device comprising a sensor and a substrate, as described above, in which is formed a microchannel in fluid communication between an entry port and an exit port. A first portion of the microchannel is configured to supply a plurality of different labeled probes, each selected to complex with one of a corresponding plurality of different analytes. A second portion of the microchannel is configured for covalently binding a plurality of corresponding different supplementary probes to the substrate. A supplementary probe of the plurality of corresponding different supplementary probes is selected to bind to a part of a corresponding analyte of the corresponding plurality of different analytes, which part is exposed when the corresponding analyte is complexed with a corresponding labeled probe of the plurality of different labeled probes. The kit also includes a supply of a plurality of molecules for each of the plurality of different labeled probes. In some embodiments, the kit includes a supply of a plurality of molecules for each of the plurality of corresponding different supplementary probes. In some embodiments, a kit also includes a reagent for covalently binding the plurality of corresponding different supplementary probes to the substrate in the second portion of the microchannel.

FIG. 5 is a block diagram that illustrates a computer system 500 upon which an embodiment of the invention, such as analyzer 190, may be implemented. Computer system 500 includes a communication mechanism such as a bus 510 for passing information between other internal and external components of the computer system 500. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). ). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 500, or a portion thereof, constitutes a means for performing one or more steps of one or more methods described herein.

A sequence of binary digits constitutes digital data that is used to represent a number or code for a character. A bus 510 includes many parallel conductors of information so that information is transferred quickly among devices coupled to the bus 510. One or more processors 502 for processing information are coupled with the bus 510. A processor 502 performs a set of operations on information. The set of operations include bringing information in from the bus 510 and placing information on the bus 510. The set of operations also typically include comparing two or more units of information, shifting positions of units of information, and combining two or more units of information, such as by addition or multiplication. A sequence of operations to be executed by the processor 502 constitute computer instructions.

Computer system 500 also includes a memory 504 coupled to bus 510. The memory 504, such as a random access memory (RAM) or other dynamic storage device, stores information including computer instructions. Dynamic memory allows information stored therein to be changed by the computer system 500. RAM allows a unit of information stored at a location called a memory address to be stored and retrieved independently of information at neighboring addresses. The memory 504 is also used by the processor 502 to store temporary values during execution of computer instructions. The computer system 500 also includes a read only memory (ROM) 506 or other static storage device coupled to the bus 510 for storing static information, including instructions, that is not changed by the computer system 500. Also coupled to bus 510 is a non-volatile (persistent) storage device 508, such as a magnetic disk or optical disk, for storing information, including instructions, that persists even when the computer system 500 is turned off or otherwise loses power.

Information, including instructions, is provided to the bus 510 for use by the processor from an external input device 512, such as a keyboard containing alphanumeric keys operated by a human user, or a sensor. A sensor detects conditions in its vicinity and transforms those detections into signals compatible with the signals used to represent information in computer system 500. Other external devices coupled to bus 510, used primarily for interacting with humans, include a display device 514, such as a cathode ray tube (CRT) or a liquid, crystal display (LCD), for presenting images, and a pointing device 516, such as a mouse or a trackball or cursor direction keys, for controlling a position of a small cursor image presented on the display 514 and issuing commands associated with graphical elements presented on the display 514.

In the illustrated embodiment, special purpose hardware, such as an application specific integrated circuit (IC) 520, is coupled to bus 510. The special purpose hardware is configured to perform operations not performed by processor 502 quickly enough for special purposes. Examples of application specific ICs include graphics accelerator cards for generating images for display 514, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 500 also includes one or more instances of a communications interface 570 coupled to bus 510. Communication interface 570 provides a two-way communication coupling to a variety of external devices that operate with their own processors, such as printers, scanners and external disks. In general the coupling is with a network link 578 that is connected to a local network 580 to which a variety of external devices with their own processors are connected. For example, communication interface 570 may be a parallel port or a serial port or a universal serial bus (USB) port on a personal computer. In some embodiments, communications interface 570 is an integrated services digital network (ISDN) card or a digital subscriber line (DSL) card or a telephone modern that provides an information communication connection to a corresponding type of telephone line. In some embodiments, a communication interface 570 is a cable modem that converts signals on bus 510 into signals for a communication connection over a coaxial cable or into optical signals for a communication connection over a fiber optic cable. As another example, communications interface 570 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 570 sends and receives electrical, acoustic or electromagnetic signals, including infrared and optical signals that carry information streams, such as digital data.

The term computer-readable medium is used herein to refer to any medium that participates in providing information to processor 502, including instructions for execution. Such a medium may take many forms, including, but not limited to, non-volatile media, volatile media and transmission media. Non-volatile media include, for example, optical or magnetic disks, such as storage device 508. Volatile media include, for example, dynamic memory 504. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 502, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 502, except for carrier waves and other signals.

Logic encoded in one or more tangible media includes one or both of processor instructions on a computer-readable storage media and special purpose hardware, such as ASIC 520.

Network link 578 typically provides information communication through one or more networks to other devices that use or process the information. For example, network link 578 may provide a connection through local network 580 to a host computer 582 or to equipment 584 operated by an Internet Service Provider (ISP). ISP equipment 584 in turn provides data communication services through the public, world-wide packet-switching communication network of networks now commonly referred to as the Internet 590. A computer called a server 592 connected to the Internet provides a service in response to information received over the Internet. For example, server 592 provides information representing video data for presentation at display 514.

The invention is related to the use of computer system 500 for implementing the techniques described herein. According to one embodiment of the invention, those techniques are performed by computer system 500 in response to processor 502 executing one or more sequences of one or more instructions contained in memory 504. Such instructions, also called software and program code, may be read into memory 504 from another computer-readable medium such as storage device 508. Execution of the sequences of instructions contained in memory 504 causes processor 502 to perform the method steps described herein. In alternative embodiments, hardware, such as application specific integrated circuit 520, may be used in place of or in combination with software to implement the invention. Thus, embodiments of the invention are not limited to any specific combination of hardware and software.

The signals transmitted over network link 578 and other networks through communications interface 570, carry information to and from computer system 500. Computer system 500 can send and receive information, including program code, through the networks 580, 590 among others, through network link 578 and communications interface 570. In an example using the Internet 590, a server 592 transmits program code for a particular application, requested by a message sent from computer 500, through Internet 590, ISP equipment 584, local network 580 and communications interface 570. The received code may be executed by processor 502 as it is received, or may be stored in storage device 508 or other non-volatile storage for later execution, or both. In this manner, computer system 500 may obtain application program code in the form of a signal on a carrier wave.

Various forms of computer readable media may be involved in carrying one or more sequence of instructions or data or both to processor 502 for execution. For example, instructions and data may initially be carried on a magnetic disk of a remote computer such as host 582. The remote computer loads the instructions and data into its dynamic memory and sends the instructions and data over a telephone line using a modem. A modem local to the computer system 500 receives the instructions and data on a telephone line and uses an infra-red transmitter to convert the instructions and data to a signal on an infra-red a carrier wave serving as the network link 578. An infrared detector serving as communications interface 570 receives the instructions and data carried in the infrared signal and places information representing the instructions and data onto bus 510. Bus 510 carries the information to memory 504 from which processor 502 retrieves and executes the instructions using some of the data sent with the instructions. The instructions and data received in memory 504 may optionally be stored on storage device 508, either before or after execution by the processor 502.

FIG. 6 illustrates a chip set 600 upon which an embodiment of the invention, such as analyzer 190, may be implemented. Chip set 600 is programmed to perform one or more steps of a method described herein and includes, for instance, the processor and memory components described with respect to FIG. 5 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 600, or a portion thereof, constitutes a means for performing one or more steps of a method described herein.

In one embodiment, the chip set 600 includes a communication mechanism such as a bus 601 for passing information among the components of the chip set 600. A processor 603 has connectivity to the bus 601 to execute instructions and process information stored in, for example, a memory 605. The processor 603 may include one or more processing cores with each core configured to perform independently. A multi-core processor enables multiprocessing within a single physical package. Examples of a multi-core processor include two, four, eight, or greater numbers of processing cores. Alternatively or in addition, the processor 603 may include one or more microprocessors configured in tandem via the bus 601 to enable independent execution of instructions, pipelining, and multithreading. The processor 603 may also be accompanied with one or more specialized components to perform certain processing functions and tasks such as one or more digital signal processors (DSP) 607, or one or more application-specific integrated circuits (ASIC) 609. A DSP 607 typically is configured to process real-world signals (e.g., sound) in real time independently of the processor 603. Similarly, an ASIC 609 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 603 and accompanying components have connectivity to the memory 605 via the bus 601. The memory 605 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 605 also stores the data associated with or generated by the execution of one or more steps of the methods described herein.

In the foregoing specification, the invention has been described with reference to specific embodiments thereof It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items. elements or steps. Furthermore, the indefinite article “a” or “an” is meant to indicate one or more of the item, element or step modified by the article.

What is claimed is:

1. A microfluidic device comprising;
a substrate in which is formed a microchannel in fluid communication between an entry port and an exit port, wherein
a first portion of the microchannel is configured to supply a plurality of different labeled probes, each selected to complex with one of a corresponding plurality of different analytes; and
a second portion of the microchannel comprises a plurality of corresponding different supplementary probes covalently bound to the substrate, wherein a supplementary probe of the plurality of corresponding different supplementary probes is selected to bind to a part of a corresponding analyte of the corresponding plurality of different analytes, which part is exposed when the corresponding analyte is complexed with a corresponding labeled probe of the plurality of different labeled probes; and
a sensor configured to detect signals emitted from the plurality of labeled probes in the second portion of the microchannel.

2. A device as recited in claim 1, further comprising an actuator configured to move a sample fluid from the entry port to the first portion of the microchannel and then to the second portion of the microchannel and then to the exit port.

3. A device as recited in claim 1, wherein the second portion of the microchannel does not overlap the first portion of the microchannel.

4. A device as recited in claim 1, wherein the first portion of the microchannel further comprises a hydrogel that encompasses the plurality of different labeled probes.

5. A method comprising:
providing a microfluidic device comprising a sensor and a substrate in which is formed a microchannel in fluid communication between an entry port and an exit port, wherein
a first portion of the microchannel is configured to supply a plurality of different labeled probes, each selected to complex with one of a corresponding plurality of different analytes, and
a second portion of the microchannel comprises a plurality of corresponding different supplementary probes covalently bound to the substrate, wherein a supplementary probe of the plurality of corresponding different supplementary probes is selected to bind to a part of a corresponding analyte of the corresponding plurality of different analytes, which part is exposed when the corresponding analyte is complexed with a corresponding labeled probe of the plurality of different labeled probes, and
the sensor is configured to detect signals emitted. from the plurality of labeled probes in the second portion of the microchannel;
moving a sample fluid from the entry port to the exit port; and
obtaining data from the sensor that indicates measurements of the signals emitted from probes bound to the supplementary probes in the second portion during an observation period.

6. A method as recited in claim 5, further comprising determining a presence of any of the plurality of analytes based on the data obtained from the sensor the sensor.

7. A method as recited in claim 5, further comprising determining at least one of a relative quantity or absolute quantity of any of the plurality of analytes based on the data obtained from the sensor.

8. A method as recited in claim 5, further comprising flushing unbound constituents from the microchannel before obtaining data from the sensor.

9. A kit comprising:
a device comprising a sensor and a substrate in which is formed a microchannel in fluid communication between an entry port and an exit port, wherein
a first portion of the microchannel is configured to supply a plurality of different labeled probes, each selected to complex with one of a corresponding plurality of different analytes, and
a second portion of the microchannel configured for covalently binding a plurality of corresponding different supplementary probes to the substrate, wherein a supplementary probe of the plurality of corresponding different supplementary probes is selected to bind to a part of a corresponding analyte of the corresponding plurality of different analytes, which part is exposed when the corresponding analyte is complexed with a corresponding labeled probe of the plurality of different labeled probes;
a supply of a plurality of molecules for each of the plurality of different labeled probes.

10. A kit as recited in claim 9, further comprising a supply of a plurality of molecules for each of the plurality of corresponding different supplementary probes.

11. A kit as recited in claim 10, further comprising a reagent for covalently binding the plurality of corresponding different supplementary probes to the substrate in the second portion of the microchannel.
